(19) United States
(12) Patent Application Publication
(10) Pub. No.: US 2019/0232324 A1
(43) Pub. Date: Aug. 1, 2019

(54) DEVICE FOR DISPENSING ATOMIZED LIQUIDS IN THE AIR

(71) Applicant: Tsafrir Sasson, Lehavot Haviva (IL)

(72) Inventor: Tsafrir Sasson, Lehavot Haviva (IL)

(21) Appl. No.: 16/331,979

(22) PCT Filed: Sep. 11, 2017
(86) PCT No.: PCT/IL2017/051023

§ 371 Date: Mar. 10, 2019

Related U.S. Application Data
(60) Provisional application No. 62/393,032, filed on Sep. 11, 2016.

(51) Int. Cl. B05B 17/06; A61L 9/14; A61L 9/12

(52) U.S. Cl. CPC B05B 17/0684; A61L 9/127; A61L 9/14

(57) ABSTRACT

A device for dispensing liquids n the air, in which a liquid atomizing module integrated into a cap of the dispensing device. The device further includes a dispensable reservoir, a wicking system for bridging the gap between said reservoir and said atomizing module. The wicking system is either a one wick or two wick liquid translocating assembly. The wick/s are made of a metal mesh. The wick having an interface with the atomizing module draws liquid from a replaceable reservoir, and the interface is stabilized by anchoring said wick to said cap.

### CROSS-REFERENCE TO RELATED APPLICATIONS

The present application is a 371 filing from PCT/IL/2017/051023, which claims the benefit of priority to U.S. Provisional Patent Application Ser. No. 62/393,032 filed Sep. 11, 2016, entitled “SYSTEM AND METHOD FOR DISPENSING LIQUIDS”. The aforementioned applications are hereby incorporated herein by reference in their entirety.

### TECHNICAL FIELD

The present invention generally relates to a wicking formation which supports the supply of liquid to a vibratory mechanism for dispensing liquids into the ambient.

### BACKGROUND ART

Liquid dispensers as described in US patent application 2011/0266359 A1 are manufactured with the understanding that while the dispensing mechanism is durable and long lasting, the liquid bearing container is disposable and replaceable to replenish the supply of liquid as it has dwindled or completely dried out.

In US patent application 2011/0266359 A1 a device for controllably dispersing liquids is disclosed. The mechanism that propels the scent bearing liquid into the ambient, employs an energized plate studded with an array of micro plugs that thrust forcibly into holes of a parallel plate such that the micro plugs can drive out the scent bearing liquid through the array of holes.

The mechanism that propels the liquid drains the liquid provided by a wick, while the wick draws the liquid from a receptacle. The wick therefore serves as a conduit of liquid from a receptacle to dispensing module. In WO publication 2015/049676 A1 a device for controllably dispersing liquids is disclosed (referred to as dispenser). The mechanism that propels the scent bearing liquid into the ambient, employs an energized plate studded with an array of micro plugs that thrust forcibly into holes of a parallel plate such that the micro plugs can drive out the scent bearing liquid through the array of holes. The liquid arrives at the atomizer 70, or more specifically at the bottom 68 of this atomizer. To make the elements of a system of the invention more clear, a description of a prior art system is provide in FIG. 1, which describes schematically the translocation of a liquid from a reservoir 32, which in a specific case is a liquid bearing scent. The liquid moves from the reservoir by capillary forces implemented by a wick 34 to a forwarding module 36 which further translocates the liquid to a dispersing module.

As can be seen in FIG. 3 of WO2015/049676, a wick 64 bridges between the liquid in the liquid reservoir and the atomizer. The wick has two ends. The distal end draws liquid from the reservoir, and the proximal end provides the same liquid to the atomizer.

In practice, liquid dispensing atomizing devices similar or the same as described in the prior art above would require replenishing of the liquid as soon as the internal stock runs out or dwindles.

### BRIEF DESCRIPTION OF THE DRAWINGS

The present invention will be understood and appreciated more fully from the following detailed description taken in conjunction with the appended drawings in which:

FIG. 1 is an isometric depiction of a dispensing device of the prior art with cap not shown as well as a sector of the liquid reservoir, exposing the wick.

FIG. 2 is a block diagram of a liquid dispenser showing the interaction between components of the flow chain with one wick employed.

FIG. 3 is a block diagram of a liquid dispenser showing the interaction between components of the flow chain with two wicks employed.

FIG. 4 is a block diagram of a liquid dispenser showing the interaction between components of the translocation train including wick packaged with cap assembly.

FIG. 5 is a block diagram of a liquid dispenser showing the interaction between components of the translocation train emphasizing the two contact zone of a two wick embodiment.

### DISCLOSURE OF THE INVENTION

Therefore, a mechanism for implementing dispensation of liquid to be employed in commercial liquid dispensing products using the technological aspects as referred to in both above citations). The object of the present invention is to facilitate a steady flow of liquid to the dispensing module.

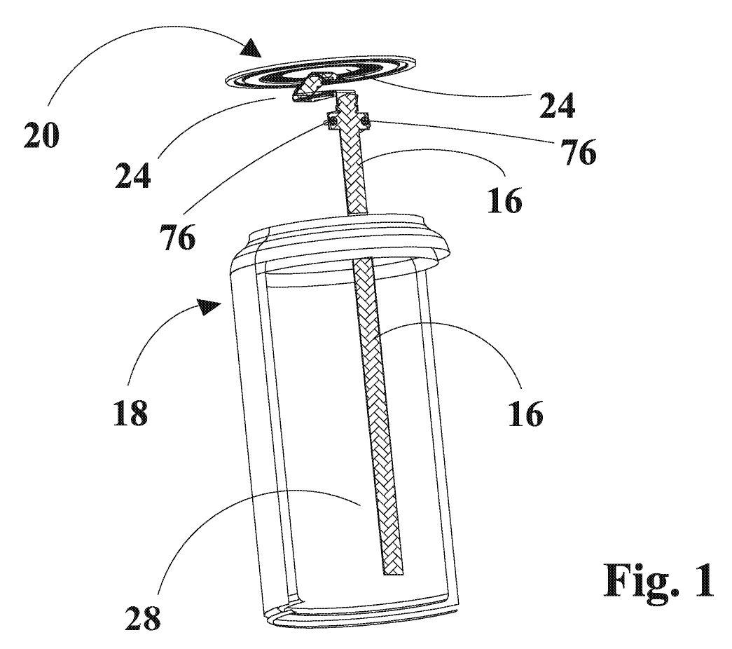

In accordance with some embodiments of the present invention a solitary wick spans the distance between the atomizer and the bottom of the liquid reservoir. As can be seen in FIG. 1, wick 16 reaches the bottom of liquid reservoir 18. At its other (upper), proximal end it reaches atomizer 20 and abuts its bottom 24.

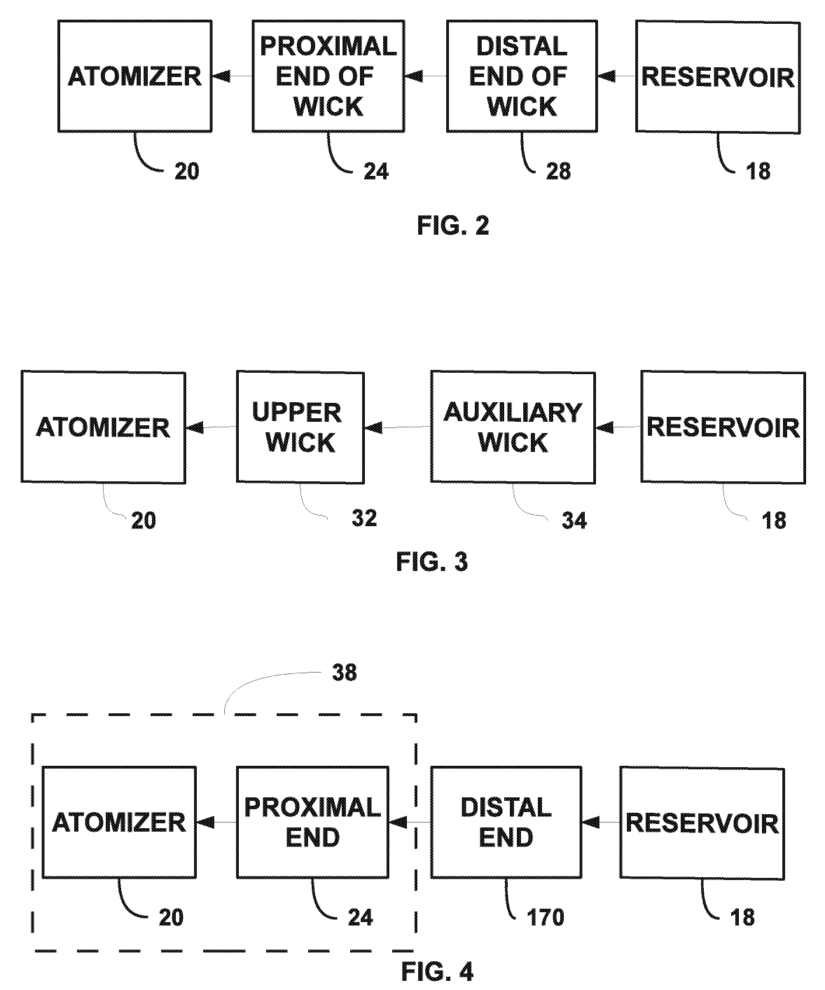

Referring to FIG. 2, Atomizer 20 draws liquid from proximal end 24 of wick 16, previously collected through the distal end 28 of the wick, that draws liquid from reservoir 18. We have realized that the exact positioning of the proximal end 24 with respect to atomizer 20 dictates the properties of the flow of liquid with high dependency on the positioning parameters.

The proximal end 24 of the wick is manufactured with strict juxtaposition parameters and low tolerance for variability of positioning with respect to the atomizer 20. The wick, and especially the proximal end 24 thereof are fixed in place relative to the atomizer, and replacement reservoirs are exchanged such that the proximal end of the wick is not moved about relative to the atomizer while replacing or afterwards. In one embodiment of the present invention, there is only one wick, as described in pictorially in FIG. 1.

The Wicking System

In accordance with the present invention either one or two wicks can be used in a functional liquid dispensing device, for bridging the gap between the liquid and the atomizing module. One wick liquid translocating assemblies (embodiments) have been described above with some details. In one wick embodiments, the replacing of an empty reservoir by a new filled up reservoir entails inserting the distal end 28 of solitary wick 16 in the new reservoir, in such a way that it is submerged in the liquid. In a different embodiment of the present invention, two wicks are functionally disposed serially in a device, such a liquid translocating assembly is better understood with reference to FIG. 3 Accordingly, in two wick assemblies (embodiments), the replacement reservoir employs an auxiliary wick. In the case of two embodiments, i.e. when an auxiliary wick is included in the replacement reservoir, a connection is formed with the distal end of the upper wick 32. Thus, the liquid is able to flow from auxiliary (lower) wick 34 to the upper wick 32. Atomizer 20 receives liquid from proximal end of upper wick 32. The upper wick is therefore in contact with auxiliary (lower) wick 34 which in turn draws the liquid from reservoir 18. In FIG. 4 a structural framework of the invention is demonstrated schematically, introducing wick fixing assembly 38. This wick fixing assembly is a substantially rigid mechanical framework which atomizer 20 is configured to be held stably with respect to proximal end 24 of the upper wick 32 or alternatively, the proximal end 24 of solitary wick 16 respectively. For example, in such a case as the atomizer forms a part of the cap of the dispenser 18 of the liquid. See page 7, explanations for FIG. 3 there, bottom paragraph in WO 2015/049676 A1.

The wick in accordance with the present invention is preferably made of a braid of metal wires/threads or a woven formation thereof, all such combinations referred to hereinafter as metal mesh that can form capillary passages between the wires to allow upward motion of the liquid from the reservoir. The wick in accordance with the present invention is a resilient body which includes attaching means such as lingules or wings to facilitate attaching the upper or solitary wick to the cap with which the atomizer 20 is associated. Such wings 76 are described in FIG. 1 to which reference is again made. A cap, not shown, into which the atomizer 20 is integrated also uses as a packaging component for harnessing the wick, be it solitary wick 16 or upper wick 32.

The wick itself is resilient for a reason that the juxtaposition against the atomizer 20 must endure some forces that otherwise could cause a disruption of the exact spatial interaction. However, the meshed wires need not necessarily be the source of springiness or not be the sole source of springiness. For example if the wires making up the mesh are soft, a resilient wire can be threaded inside a tubular wick to convey a stronger force to the wick, i.e., increase the spring force.

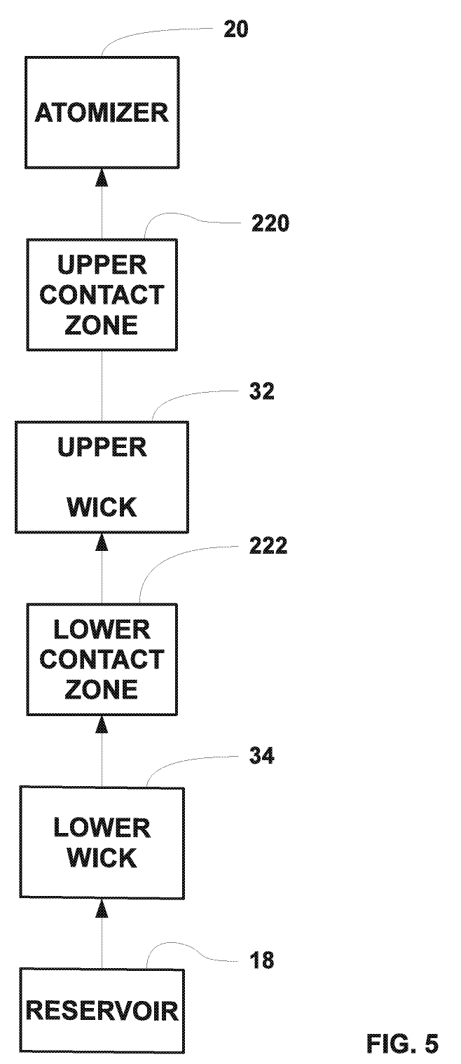

In a two wick embodiment there are two contact zones involved with the liquid translocation train. As can be seen in FIG. 5, in a two wick assembly of a dispensing device, there are two contact zones involved in the translocation train of liquid. One upper contact zone 220, which is the interface between atomizer 20 and upper wick 32. The lower contact zone 222.

Within the contact zone, the wick or wicks can be shaped in a form that would enlarge the surface of interaction, such as bending the fashion describe pictorially in FIG. 1 for the proximal zone (end) 24. In the interface zone involving the two wicks, which is referred to above as lower contact zone 222, the two wicks can have their contact area enlarged by having them bent and set one against the other accordingly.

1. A device for dispensing liquids in the air, said device comprising:
a liquid atomizing module integrated into a cap of said dispensing device;
a dispensable reservoir,
a wicking system for bridging the gap between said reservoir and said atomizing module, including at most two wicks, wherein said at most two wicks are made at least of a metal mesh;
whereby the wick having an interface with said atomizing module draws liquid from a replaceable reservoir, and wherein said interface is stabilized by anchoring said wick to said cap.

2. The device as in claim 1 wherein at least one of said at most two wicks contain a resilient threaded wire for promoting resilience to said at least one wick.

3. The device as in claim 1 wherein a wick fixing assembly is introduced, in which the proximal end of the wick having an interface with said atomizing module and said atomizing module form a substantially rigid mechanical framework.

4. The device as in claim 1 wherein said wick fixing assembly is implemented with one wick liquid translocating assembly.

5. The device as in claim 1 wherein said wick fixing assembly is implemented with a two wick liquid translocating assembly.
